(19) United States
(12) Patent Application Publication
(10) Pub. No.: US 2006/0147546 A1
(43) Pub. Date: Jul. 6, 2006

(54) Compound consisting of precipitated silica and phosphate and use thereof as nutrient intake liquid support and as anticaking agent with nutrient intake

(76) Inventors: Patrick Ferlin, Paris (FR); Lorraine Leite, Paris (FR); Pierre-Yves Lahary, Lyon (FR); Remi Valero, Lyon (FR)

Correspondence Address: Jean-Louis Seugnet;Rhodia Inc, Intellectual Property Department, 259 Prospect Plains Road CN 7500, Cranbury, NJ, 08512-7500, US

(21) Appl. No.: 10/525,107

(22) PCT Filed: Aug. 21, 2003
(86) PCT No.: PCT/FR03/02560

§ 371 Date: Sep. 22, 2005

(30) Foreign Application Priority Data
Aug. 30, 2002 (FR) 02/10836

(51) Int. Cl. A61K 33/42

(52) U.S. Cl. 424/601; 424/603

(57) ABSTRACT

The invention concerns compounds, for use as liquid support and as anticaking agent, and, simultaneously, as nutrient additive in particular for animals, consisting of precipitated silica and phosphate selected among phosphates of elements of groups Ia or IIa of the periodic table of the elements and rare-earth phosphates, said compounds being in the form of substantially spherical pellets.

The present invention relates to novel compounds based on precipitated silica and phosphate, in particular calcium phosphate, for use as a support for a liquid, in particular a liquid animal feedstuff complement, and preferably simultaneously as a nutritional additive, in particular for animals.

It also relates to compositions comprising a liquid, in particular a liquid animal feedstuff complement, absorbed on a support formed by said novel compound based on precipitated silica and phosphate.

Finally, the present invention also relates to the use of said compounds, preferably after milling, as an anticaking agent, a liquid atomization processing aid, a solid milling or pelletization/tabletting processing aid, and preferably simultaneously as a nutritional additive for animals.

It is known for liquids, more particularly animal feedstuff additives, to be conditioned on solid supports, in particular on a silica support. Said conditioning is generally aimed at transforming a liquid that cannot be handled or is difficult to handle into a fluid powder that can easily be stored, for example in sacks or loose, which is easier to handle and which also can readily be dispersed and mixed with other divided solid constituents.

In the following text, the term “conditioned composition” means the composition obtained, i.e. a liquid absorbed on a silica support.

Said conditioned composition must be capable of being handled easily, which implies good fluidity and low powdering, and hence the support has to be mechanically strong and have good resistance to wear. It also has to have a relatively high active material (liquid) content and hence the support has to have a high absorption capacity and also a relatively high density. Those different requirements are sometimes contradictory and are not necessarily satisfied by prior art silica supports.

The aim of the invention is to provide novel compounds constituting an alternative to known silica supports and which are particularly suitable for conditioning liquids, especially liquid animal feedstuff complements.

To this end, in one aspect the invention provides a compound (or mixture) that can be obtained by spray drying a suspension hereinafter designated S containing a precipitated silica and a phosphate selected from phosphates of elements from groups Ia or IIa of the periodic table of the elements and rare earth phosphates.

Whether the precipitated silica is used as is in its solid form or in the form of an aqueous suspension obtained by re-dispersing the precipitated silica in the solid form in water, the precipitated silica is very advantageously employed in the form of a filter cake or a suspension directly from its preparation process (precipitation reaction).

To this end, the present invention also proposes a compound (or mixture) formed from precipitated silica and at least one phosphate selected from phosphates of elements from groups Ia or IIa of the periodic table of the elements and rare earth phosphates.

In accordance with the invention, drying is carried out by atomization (co-atomization), i.e. by spraying the suspension S into a hot atmosphere (spray drying). The compound (or mixture) of the invention can be termed a “co-atomizate”. Drying is advantageously carried out using an atomizing nozzle, for example monofluid or pressurized liquid. The temperature at the outlet from the atomizer is normally less than 170° C., in particular less than 140° C.; as an example, it is in the range 100° C. to 135° C.

Preferably, immediately prior to drying, the dry matter content of the suspension S is in the range 16% to 24% by weight, in particular in the range 18% to 24% by weight, for example in the range 18% to 22% by weight.

In a variation of the invention, the suspension S is obtained by mixing two precursors of a phosphate selected from phosphates of elements from groups Ia or IIa of the periodic table of the elements and rare earth phosphates with a suspension of precipitated silica. The term “two precursors” of a phosphate means a precursor providing the phosphate “portion” per se selected, for example, from orthophosphoric acid H₃PO₄ and its salts with formulae NH₄H₂PO₄, NaH₂PO₄, KH₂PO₄, (NH₄)₂HPO₄, and a precursor providing the element from group Ia or group IIa of the periodic table of the elements or rare earth “portion” selected in the case of calcium, for example, from lime Ca(OH)₂, calcium nitrate Ca(NO₃)₂ and calcium chloride CaCl₂.

In general, in this variation, the two phosphate precursors are added to the suspension of precipitated silica, usually with agitation, each in the solid form (i.e. the dry form, in particular a powder) or, as is preferable, in the form of an aqueous solution (which includes the case in which one is added in the solid form and the other is added in the form of a solution), under conditions such that said phosphate selected from phosphates of elements from groups Ia or IIa of the periodic table of the elements and rare earth phosphates, is formed. The two precursors of said phosphate can be added simultaneously to the suspension of precipitated silica; preferably, they are added successively, the precursor providing the phosphate “portion” per se being added first.

The mixture obtained can optionally undergo a disintegration operation, which can if necessary reduce the viscosity of the suspension to be dried subsequently. The disintegration operation can in particular be carried out by passing the mixture into a mill, in particular of the colloidal or ball type or, as is preferable, into a high shear agitator, for example in the presence of water. It should be noted that the disintegration operation may coincide with the mixing operation.

Mixing and optional disintegration are generally carried out at a temperature in the range 15° C. to 70° C., for example in the range 20° C. to 50° C.

In this variation, the suspension of precipitated silica initially used may be directly from a process for preparing precipitated silica or may be obtained by disintegrating a filter cake from said preparation process (precipitation reaction). Said precipitated silica suspension generally has a dry matter content in the range 16% to 24% by weight, in particular in the range 18% to 24% by weight, for example in the range 18% to 22% by weight.

In a further variation of the invention, the suspension S is obtained by mixing, generally with agitation, either a precipitated silica constituted by a filter cake from the reaction for precipitating said silica, or a suspension of precipitated silica, preferably obtained by disintegrating a filter cake from the reaction for precipitating said silica, with a phosphate selected from phosphates of elements from groups Ia or IIa of the periodic table of the elements and rare earth phosphates.

The filter cake disintegration operation can more particularly reduce its viscosity and can in particular be carried out by passing the cake through a high shear mixer or a mill, in particular of the colloidal or ball type, for example in the presence of water, and preferably in the presence of an aluminium compound, in particular sodium aluminate.

Similarly, the mixture obtained formed either from precipitated silica constituted by the filter cake or from the precipitated silica suspension and from the phosphate can optionally undergo a disintegration operation, which can reduce its viscosity if necessary. The disintegration operation can in particular be carried out by passing the mixture through a mill, in particular a colloidal or ball type mill or, as is preferable, through a high shear mixer, for example in the presence of water. It should be noted that the disintegration operation may coincide with the mixing operation. en precipitated silica constituted by a filter cake is used, a disintegration operation is generally carried out.

Mixing and optional disintegration are generally carried out at a temperature in the range 15° C. to 70° C., for example in the range 20° C. to 50° C.

The phosphate can be used in the form of an aqueous suspension or in the solid form, (for example granules or, as is preferable, as a powder), with water optionally also being added to the precipitated silica suspension, generally with agitation.

In this variation, the possible suspension of the precipitated silica used initially generally has a dry matter content in the range 16% to 24% by weight, in particular in the range 18% to 24% by weight, for example in the range 18% to 22% by weight.

Finally, although not constituting a preferred variation of the invention, the suspension S can optionally be obtained by mixing precipitated silica in the solid form with a solution of a phosphate selected from phosphates of elements from groups Ia or IIa of the periodic table of the elements and rare earth phosphates.

The precipitated silica, in particular in the form of a suspension or a filter cake, used in accordance with the invention, is preferably prepared by a process of the type comprising reacting a silicate with an acidifying agent then carrying out an optional separation operation (liquid-solid separation), the silica precipitation being carried out as follows:
(1) forming an initial stock comprising at least a portion of the total quantity of silicate engaged in the reaction and in general at least one electrolyte, the concentration of silicate (expressed as SiO₂) in said initial stock being less than 100 g/l, in particular less than 90 g/l, and the concentration of electrolyte (for example sodium sulphate) in said initial stock being less than 17 g/l, for example less than 14 g/l;
(2) adding acidifying agent to said stock to obtain a pH in the reaction medium of at least about 7, generally in the range about 7 to 8;
(3) adding acidifying agent to the reaction medium simultaneously with the remaining quantity of silicate, if appropriate.

It should be noted that in general, the process concerned is a process for synthesizing a precipitated silica, i.e. an acidifying agent is caused to act on a silicate under particular conditions.

The choice of acidifying agent and silicate are made in a manner that is known per se.

Generally, the acidifying agent used is a strong mineral acid such as sulphuric acid, nitric acid or hydrochloric acid, or an organic acid such as acetic acid, formic acid or carbonic acid.

The acidifying agent can be dilute or concentrated; its normality can be in the range 0.4 to 36 N, for example in the range 0.6 to 1.5 N.

Particularly in the case in which the acidifying agent is sulphuric acid, its concentration can be in the range 40 to 180 g/l, for example in the range 60 to 130 g/l.

It is also possible to use as the silicate any normal form of silicate such as metasilicates, disilicates or, advantageously, an alkali metal silicate, in particular sodium or potassium silicate.

The silicate can have a concentration (expressed as SiO₂) in the range 40 to 330 g/l, for example in the range 60 to 300 g/l, in particular in the range 60 to 260 g/l.

In general, the acidifying agent used is sulphuric acid and the silicate is sodium silicate. When using sodium silicate, it generally has a SiO₂/Na₂O weight ratio in the range 2 to 4, for example in the range 3.0 to 3.8.

The initial stock generally comprises an electrolyte. The term “electrolyte” as used here has its normal meaning, i.e. it means any ionic or molecular substance which, when in solution, decomposes or dissociates to form ions or charged particles. Electrolytes which can be cited include salts from the alkali and alkaline-earth metal salt group, more particularly the salt of the starting metal silicate and the acidifying agent, for example sodium chloride when reacting a sodium silicate with hydrochloric acid or, as is preferable, sodium sulphate when reacting a sodium silicate with sulphuric acid.

In the (preferred) case in which the starting stock comprises only a portion of the total quantity of silicate engaged in the reaction, the acidifying agent and the remaining quantity of silicate are simultaneously added in step (3).

Said simultaneous addition is preferably carried out in a manner that keeps the pH to a value that is equal to (± 0.2) the value at the end of step (2).

In general, in a subsequent step, a supplemental quantity of acidifying agent is added to the reaction medium until the pH of the reaction medium is in the range 3 to 6.5, in particular in the range 4 to 6.5.

It may then be advantageous to mature the reaction medium, following said addition of a supplemental quantity of acidifying agent, said maturing taking 2 to 60 minutes, in particular 3 to 20 minutes, for example.

When the starting stock comprises the total quantity of the silicate used in the reaction, in step (3), the acidifying agent is added, preferably until the pH of the reaction medium reaches a value in the range 3 to 6.5, in particular in the range 4 to 6.5.

It may then be advantageous to mature the reaction medium following said step (3), said maturing taking 2 to 60 minutes, more particularly 3 to 20 minutes, for example.

The reaction chamber in which the complete reaction of the silicate with the acidifying agent is carried out is normally provided with suitable agitation equipment and heating equipment.

The complete reaction of the silicate with the acidifying agent is generally carried out between 70° C. and 98° C.

In a variation of the process, the complete reaction of the silicate with the acidifying agent is carried out at a constant temperature, preferably in the range 80° C. to 95° C.

In a (preferred) variation of the process, the temperature at the end of the reaction is higher than the temperature at the start of the reaction: thus, the temperature at the start of the reaction is preferably kept between 70° C. and 95° C., then the temperature is increased, preferably to a value in the range 80° C. to 98° C., and it is kept at that value until the end of the reaction.

On completion of the steps described above, a slurry/suspension of silica is obtained, which can then undergo a liquid-solid separation operation.

In general, said separation comprises filtering and washing using a filter provided with compacting means.

Said filter can be a band filter provided with a roller for compacting.

However, said filter is preferably a filter press; separation then generally comprises filtering, washing then compacting using said filter.

The phosphate employed in the context of the invention is selected from phosphates of elements from groups Ia or IIa of the periodic table of the elements and rare earth phosphates.

It is generally selected from phosphates of sodium, potassium, calcium, magnesium and rare earths (more particularly cerium, lanthanum). Preferably, said phosphate is a calcium phosphate, in particular a monocalcium phosphate (MCP) also known as calcium dihydrogen phosphate with formula Ca(H₂PO₄)₂, dicalcium phosphate (DCP) also known as calcium hydrogen phosphate CaHPO₄, or a tricalcium phosphate (TCP) also known as hydroxyapatite; highly preferably, a monocalcium phosphate (MCP) or a dicalcium phosphate (DCP) is used.

The phosphates used generally have a median particle size d₅₀ of less than 100 μm, in particular less than 50 μm, and more particularly less than 25 μm.

The compounds of the invention can optionally undergo a subsequent heat treatment.

In the following description, the tamped packing density (TPD) and the non tamped packing density (NPD) were determined in accordance with French standard NF T 30-042.

The DOP oil uptake was measured in accordance with NF T 30-022 (March 1953) using dioctylphthalate.

The pore volumes given were measured by mercury porosimetry; each sample was prepared as follows: each sample was initially oven dried for 2 hours at 200° C., then placed in a test receptacle during the 5 minutes following withdrawal from the oven and then vacuum degassed, for example using a rotary vane pump; the pore diameters (MICROMERITICS Autopore III 9420 porosimeter) were calculated using the WASHBURN relationship with a contact angle theta of 140° and a surface tension gamma of 484 dynes/cm (or N/m).

The BET specific surface area was determined using the BRUNAUER-EMMET-TELLER method described in the “Journal of the American Chemical Society”, vol. 60, page 309, February 1938 and corresponded to International standard ISO 5794/1 (annex D).

The CTAB specific surface area was the external surface area determined in accordance with French standard NF T 45007 (November 1987) (5.12).

The Carr index (Ci) of the compounds of the invention, which illustrates their fluidity (flowability), was determined using the following relationship: Ci=(TPD-NPD)/TPD.

The wear resistance of the compounds of the invention was determined as follows: it was expressed as the percentage of particles in a 100 μm−200 μm cut obtained by sieving remaining after wear lasting 2 minutes (wear resistance designated R_{wr2}), 5 minutes (wear resistance designated R_{wr5}), and 10 minutes (wear resistance designated R_{wr10}), on a 50 μm vibrating sieve in the presence of 50 glass beads with a diameter of 4 mm, the initial mass of the sample particles initially disposed on the vibrating sieve being 1 gram. During the wear procedure, the sieve was vibrated using a RETSCH VE 1000 vibrating table used with a 2 mm amplitude.

The median diameter d₅₀ (by weight) was determined using a MALVERN Mastersizer 2000 and its Hydro 2000 G suspension sampler.

The compounds (or mixtures) of the invention generally have an amount of phosphate, selected from phosphates of elements from groups Ia or IIa of the periodic table of the elements and rare earth phosphates, of at least 10% by weight, preferably at least 20% by weight (dry weight). Advantageously, their phosphate content is in the range 20% to 60% by weight, in particular in the range 20% to 50% by weight. In particular, it can be between 20% and 40% by weight, for example between 20% and 35% by weight.

The compounds of the invention are advantageously in a particular form, namely in the form of substantially spherical beads with a median diameter d₅₀ which is generally at least 80 μm, preferably at least 100 μm; said diameter is, for example, in the range 100 μm to 400 μm, more particularly in the range 110 μm to 300 μm and in particular in the range 130 μm and 280 μm. Said beads generally have a sphericity factor (defined as indicated in International patent application WO-A-98/35751, a value of 1 corresponding to a perfect sphere) of at least 0.900, in particular at least 0.920, for example at least 0.940. Their sphericity factor can be at least 0.960. Preferably, said beads are solid (i.e. not hollow) and are not powdery, i.e. generate little or no dust especially during handling.

The compounds of the invention advantageously have good mechanical resistance/cohesion in particular good wear resistance, which ensures their non powdery character especially during handling and also a porosity that provides them with a high absorbing power.

In general, they thus have:
a wear resistance R_{wr2} of at least 60%, in particular at least 80%, more particularly at least 82%; and/or
a wear resistance R_{wr5} of at least 50%, in particular at least 55%; and/or
a wear resistance R_{wr10} of at least 15% in particular at least 17%.

Their DOP oil uptake is normally more than 170 ml/100 g, more particularly more than 210 ml/100 g. It may be at least 230 ml/100 g, for example at least 240 ml/100 g.

The compounds of the invention advantageously have a DOP oil uptake that is higher than the DOP oil uptake of the composition obtained by dry mixing said precipitated silica in the solid form with said phosphate in the solid form.

Their pore volume (V_{d1}) constituted by pores with a diameter of less than 1 μm can be at least 1.2 cm³/g, in particular at least 1.3 cm³/g, more particularly at least 1.4 cm³/g; as an example, it can be at least 1.5 cm³/g. It is generally less than 2.2 cm³/g, for example 1.8 cm³/g.

The compounds of the invention have a fairly high density, more particularly higher than that of the precipitated silica they contain; their tamped packing density (TPD) is preferably more than 0.29, in particular at least 0.30. It can be at least 0.31, for example at least 0.33.

Their PET specific surface area is generally in the range 60 m²/g to 250 m²/g, in particular in the range 90 m²/g to 200 m²/g, for example in the range 100 m²/g to 160 m²/g.

They have very good fluidity (flowability), which in general is improved over that of the precipitated silica they contain. They may have a Carr index (Ci) of less than 0.1.

The Applicant has discovered that the compounds (or mixtures) defined above advantageously have a high absorption capacity, improved fluidity and good mechanical resistance/cohesion, in particular good wear resistance, resulting in a non powdery character, especially during handling, and are particularly suitable for conditioning liquids.

In further aspects, the invention pertains to the use of a compound as described above as a support for a liquid and to a conditioned composition comprising at least one liquid absorbed on a support formed by a compound as defined above.

Liquids that can be cited are organic liquids such as organic acids, surfactants, for example of the anionic or non-ionic type, organic additives for rubber/polymers, or pesticides.

However, particular examples of liquids that can be used here are liquid additives such as: preservatives (more particularly phosphoric acid, propionic acid), flavours, colorants, liquid foodstuff complements.

The compounds described above are particularly suitable for conditioning liquid foodstuff complements, more particularly liquid animal foodstuff complements. Examples that can be cited are choline, choline hydrochloride, vitamins such as vitamins A, B, C, D, K, and, preferably, vitamin E (or its acetate).

One essential advantage of the present invention resides in the fact that in addition to their use as a support for a liquid additive, in particular for a liquid animal foodstuff complement, the compounds of the invention have a nutritional value, or even a therapeutic value and can be used simultaneously as a nutritional or even a therapeutic additive for animals, thus encouraging the growth and health of animals, more particularly breeding animals.

The present invention associates, in one and the same product, a nutritional additive or even therapeutic additive such as calcium phosphate with a liquid additive, more particularly a liquid foodstuff complement, in particular animal foodstuff, such as vitamin E (or its acetate), for example.

The operation for absorbing liquid onto the support formed by the compound of the invention can be carried out conventionally, in particular by spraying the liquid onto the support in a mixer.

The conditioned composition of the invention can, more particularly in the case of vitamin E (or its acetate), have a liquid content of at least 50% by weight, in particular in the range 50% to 70%, for example in the range 50% to 65% by weight; the liquid content can be at least 52% by weight. This high liquid content illustrates the high absorbing power with which the compounds of the invention are preferentially endowed. Even higher liquid contents can be employed, in particular in the case of choline hydrochloride.

It should be noted that the compounds of the invention can permit more rapid and/or easier release of a liquid, more particularly of vitamin E (or its acetate) into its medium of use, for example the body of an animal.

Because of the presence of the compound described above, the conditioned compositions of the invention preferably exhibit little or no dust formation and very good fluidity (flowability), combined with a rather high density.

The present invention also pertains to the use of the compounds of the invention as an anticaking agent; preferably, said compounds are milled prior to said use, for example to a particle size in the range 1 μm to 100 μm, more particularly in the range 2 μm to 50 μm. They can be used as an anticaking agent in human foodstuffs, for example fishes, cheeses, sugar, polydextrose, flavours, dried fruits, coffee powder, tea, cocoa, in animal foodstuffs, for example formulations, feeds, and also in agriculture, in the detergent industry, in the pharmacy, in cosmetics and in a variety of industrial applications (such as rubber/polymers, toners, fire extinguisher powder, concrete, latex powder).

It also concerns their use as a liquid atomization processing aid, as a solid milling processing aid, and in particular in the detergent industry and in the pharmacy as a pelletization and/or tabletting aid; preferably, said compounds are milled prior to said use, for example to a particle size in the range 1 μm to 100 μm, in particular in the range 2 μm to 50 μm.

When used as a liquid atomization aid, when added to the liquid that is to be dried by atomization, it prevents adhesion to the walls of the atomizer and also produces a non-caked final powder with good flowability (possible application: the dairy fattening industry).

When used as a powder milling aid, when added to a powder in a mill, it improves milling of said powder and also produces a final non-caked powder with good flowability (possible application: polymer industry).

When used as an anticaking agent, preferably after milling, a liquid atomization processing aid, as a processing aid for milling a solid or for pelletization/tabletting, the compounds of the invention have the major advantage of having a nutritional value and of being able to be used simultaneously as a nutritional additive, more particularly for animals.

The following examples illustrate the invention without in any way limiting the invention.

### EXAMPLE 1

1) The following were introduced into a stainless steel reactor provided with a propeller agitator system and jacket heating system:
   345 litres of water;
   7.5 kg of Na₂SO₄;
   588 litres of aqueous sodium silicate with a SiO₂/Na₂O weight ratio of 3.5 and a density at 20° C. of 1.133.

The silicate concentration, expressed as SiO₂, in the initial stock was thus 85 g/l. The mixture was heated to a temperature of 82° C., maintaining agitation. 387 litres of dilute sulphuric acid with a density at 20° C. of 1.050 was introduced to obtain in the reaction medium a pH (measured at its temperature) of 8.0. The reaction temperature was 82° C. for the first 25 minutes; it was then heated from 82° C. to 92° C. over 15 minutes, then kept at 92° C. until the end of the reaction.

82 litres of aqueous sodium silicate of the type described above and 134 litres of sulphuric acid, also of the type described above, were then introduced (i.e. when the pH of the reaction medium had reached 8.0) together into the reaction medium, said simultaneous introduction of acid and silicate being carried out in a manner such that the pH of the reaction medium during the introduction period was kept at 8.0±0.1. After introducing all of the silicate, dilute acid introduction was continued for 9 minutes to bring the pH of the reaction medium to 5.2. After said acid introduction, the reaction slurry obtained was agitated for 5 minutes.

The total reaction period was 118 minutes.

A slurry or suspension of precipitated silica was obtained, which was then filtered and washed using a vertical plate filter press (said plates being provided with deformable membranes which could compress the filter cake by introducing pressurized air), at a pressure of 4.5 bars and for the time required to obtain a cake of silica with a loss on ignition of 80.5% (and thus a dry matter content of 19.5% by weight).

The cake obtained was then fluidized by mechanical and chemical action (adding a quantity of sodium aluminate corresponding to a Al/SiO₂ weight ratio of 3000 ppm); during said operation, water was added to obtain a slurry with a loss on ignition of 81.0% (and thus a dry matter content of 19.0% by weight). After this disintegration operation, the resulting suspension R, with a pH of 6.4, was dried using a monofluid nozzle atomizer.

The precipitated silica obtained was in the form of substantially spherical beads and had the following characteristics:

| BET specific surface area | 159 m²/g |
|---|---|
| Median diameter d₅₀ | 174 μm |
| DOP oil uptake | 296 ml/100 g |
| Pore volume (V_{d1}) constituted | 2.0 cm³/g |
| by pores with d < 1 μm | |
| TPD | 0.27 |
| NPD | 0.24 |
| Carr index Ci | 0.111 |
| Wear resistance | |
| R_{wr2} | 83% |
| R_{wr5} | 56% |
| R_{wr10} | 18% |

2) Vitamin E acetate was placed on the support formed by the silica prepared in 1).

The vitamin E acetate was supported in a 7 litre Patterson Kelley V mixer rotating at 20 rpm with an internal axis rotating at 1900 rpm provided with plates through which the vitamin E acetate was sprayed and on which lump breaking knives were mounted.

800 g of the silica prepared in 1) was charged into the mixer, then 978 g of vitamin E acetate was sprayed at a temperature of 80° C. for 10 minutes over said silica. Agitation in the homogenizer was maintained for 5 more minutes.

The conditioned composition obtained contained 45% by weight of precipitated silica and 55% by weight of vitamin E acetate and had the following supplemental characteristics:

| | |
|---|---|
| TPD | 0.58 |
| NPD | 0.53 |
| Carr index Ci | 0.086 |

### EXAMPLE 2

1) 156 kg of a suspension of precipitated silica R as prepared in 1) with a dry matter content of 19.0% by weight was charged into a 300 litre stainless steel tank provided with a blade agitator. Said suspension was pumped and sent through a circuit to a 60 litre reactor provided with a triple blade agitator. 10 kg of monocalcium phosphate powder (i.e. 25% by weight of calcium phosphate with respect to the dry weight of calcium phosphate+silica) sold under the trade name IBEX^{*} MCP by Rhodia Consumer Specialities was added to said suspension in the reactor (which was at a temperature of about 20° C.) using a worm dosimeter, along with 40 kg of water; the introduction period was about 1 hour. The resulting suspension was then dried using a monofluid nozzle atomizer.

The compound obtained, formed from precipitated silica and calcium phosphate, was in the form of substantially spherical beads and had the following characteristics:

| BET specific surface area | 103 m²/g |
|---|---|
| Median diameter d₅₀ | 136 μm |
| DOP oil uptake | 241 ml/100 g |
| Pore volume (V_{d1}) constituted | 1.7 cm³/g |
| by pores with d < 1 μm | |
| TPD | 0.33 |
| NPD | 0.30 |
| Carr index Ci | 0.091 |
| Wear resistance | |
| R_{wr2} | 84% |
| R_{wr5} | 57% |
| R_{wr10} | 25% |

This compound of the invention was thus denser than the precipitated silica obtained in Example 1. Its fluidity was also improved (lower Carr index) and its wear resistance was better, all the while having nutritional properties.
2) Vitamin E acetate was placed on the support formed by the compound (mixed silica-phosphate) prepared in 1).

The vitamin E acetate was supported in a 7 litre Patterson Kelley V mixer rotating at 20 rpm with an internal axis rotating at 1900 rpm provided with plates through which the vitamin E acetate was sprayed and on which lump breaking knives were mounted.

1000 g of the compound prepared in 1) was charged into the mixer, then 1222 g of vitamin E acetate was sprayed at a temperature of 80° C. for 10 minutes over said compound. Agitation in the homogenizer was maintained for 5 more minutes.

The conditioned composition obtained contained 45% by weight of precipitated silica and 55% by weight of vitamin E acetate and had the following supplemental characteristics:

| | |
|---|---|
| TPD | 0.71 |
| NPD | 0.65 |
| Carr index Ci | 0.084 |

This conditioned composition, based on a mixed silica-phosphate support in the form of substantially spherical beads, had good fluidity, as illustrated by the low Carr index, said fluidity even being improved over that of the conditioned composition prepared in Example 1. Its density was also higher.

### EXAMPLE 3

1) 156 kg of a suspension of precipitated silica R as prepared in 1) with a dry matter content of 19.0% by weight was charged into a 300 litre stainless steel tank provided with a blade agitator. Said suspension was pumped and sent through a circuit to a 60 litre reactor provided with a triple blade agitator. 9.9 kg of tricalcium phosphate powder (i.e. 25% by weight of calcium phosphate with respect to the dry weight of calcium phosphate+silica) sold under the trade name TCP 118 FG by Rhodia Consumer Specialities was added to said suspension in the reactor (which was at a temperature of about 20° C.) using a worm dosimeter, along with 41 kg of water; the introduction period was about 1 hour. The resulting suspension was then dried using a monofluid nozzle atomizer.

The compound obtained, formed from precipitated silica and calcium phosphate, was in the form of substantially spherical beads and had the following characteristics:

| BET specific surface area | 129 m²/g |
|---|---|
| Median diameter d₅₀ | 154 μm |
| DOP oil uptake | 252 ml/100 g |
| Pore volume (V_{d1}) constituted | 1.6 cm³/g |
| by pores with d < 1 μm | |
| TPD | 0.31 |
| NPD | 0.28 |
| Carr index Ci | 0.097 |
| Wear resistance | |
| R_{wr2} | 84% |
| R_{wr5} | 56% |
| R_{wr10} | 19% |

This compound of the invention was thus denser than the precipitated silica obtained in Example 1. Its fluidity was also improved (lower Carr index) and its wear resistance was better, all the while having nutritional properties.
2) Vitamin E acetate was placed on the support formed by the compound (mixed silica-phosphate) prepared in 1).

The vitamin E acetate was supported in a 7 litre Patterson Kelley V mixer rotating at 20 rpm with an internal axis rotating at 1900 rpm provided with plates through which the vitamin E acetate was sprayed and on which lump breaking knives were mounted.

900 g of the silica prepared in 1) was charged into the mixer, then 1100 g of vitamin E acetate was sprayed at a temperature of 80° C. for 10 minutes over said silica. Agitation in the homogenizer was maintained for 5 more minutes.

The conditioned composition obtained contained 45% by weight of mixed silica-phosphate and 55% by weight of vitamin E acetate and had the following supplemental characteristics:

| | |
|---|---|
| TPD | 0.70 |
| NPD | 0.64 |
| Carr index Ci | 0.0857 |

This conditioned composition based on a mixed silica-phosphate support in the form of substantially spherical beads had good fluidity, as illustrated by the low Carr index. Its density was higher than that of the conditioned composition prepared in Example 1.

### EXAMPLE 4

1) 178 kg of a suspension of precipitated silica R as prepared in 1) with a dry matter content of 19.0% by weight was charged into a 300 litre stainless steel tank provided with a blade agitator. Said suspension was pumped and sent through a circuit to a 60 litre reactor provided with a triple blade agitator. 22.3 kg of tricalcium phosphate powder (i.e. 40% by weight of calcium phosphate with respect to the dry weight of calcium phosphate+silica) sold under the trade name TCP 118 FG by Rhodia Consumer Specialities was added to said suspension in the reactor (which was at a temperature of about 20° C.) using a worm dosimeter, along with 41 kg of water; the introduction period was about 1 hour. The resulting suspension was then dried using a monofluid nozzle atomizer.

The compound obtained, formed from precipitated silica and calcium phosphate, was in the form of substantially spherical beads and had the following characteristics:

| BET specific surface area | 112 m²/g |
|---|---|
| Median diameter d₅₀ | 144 μm |
| DOP oil uptake | 240 ml/100 g |
| Pore volume (V_{d1}) constituted | 1.5 cm³/g |
| by pores with d < 1 μm | |
| TPD | 0.36 |
| NPD | 0.33 |
| Carr index Ci | 0.083 |
| Wear resistance | |
| R_{wr2} | 83% |
| R_{wr5} | 55% |
| R_{wr10} | 18% |

This compound of the invention was thus denser than the precipitated silica obtained in Example 1. Its fluidity was also greatly improved (much lower Carr index) and its wear resistance was still satisfactory, all the while having nutritional properties.

1-37. (canceled)

38. A compound made by the process comprising the steps of:
a) spray drying a suspension S containing a precipitated silica and a phosphate of an element selected from the group consisting of group Ia of the periodic table of the elements, group IIa of the periodic table of the elements and a rare earth; and
b) recovering the compound obtained in step a).

39. The compound according to claim 38, wherein in step a) said suspension S is obtained by mixing two precursors of the phosphate with a suspension of precipitated silica, and by, optionally, disintegrating the mixture obtained.

40. The compound according to claim 39, wherein said precipitated silica suspension is obtained by disintegrating a filter cake obtained from a reaction for precipitating said silica.

41. The compound according to claim 39, wherein said precipitated silica suspension has a dry matter content in a range of 16% to 24% by weight.

42. The compound according to claim 39, wherein in step a), the two phosphate precursors are added to said precipitated silica suspension, each in a solid form or in a form of an aqueous solution, under conditions such that said phosphate is formed, the precursor supplying the phosphate portion per se optionally being added first.

43. The compound according to claim 38, wherein said suspension S of step a) is obtained by mixing either a precipitated silica constituted by a filter cake from a reaction for precipitating said silica, or a suspension of precipitated silica obtained by disintegrating a filter cake from the reaction for precipitating said silica, with the phosphate, and step b) optionally comprised disintegrating the mixture obtained in step a).

44. The compound according to claim 43, wherein said suspension of precipitated silica has a dry matter content in a range of 16% to 24% by weight.

45. The compound according to claim 43, wherein said phosphate is added in a solid form, water also optionally being further added.

46. The compound according to claim 43, wherein said phosphate is added in the form of a suspension.

47. The compound according to claim 38, wherein said suspension S has a dry matter content in the range of 16% to 24% by weight, immediately before spray drying it.

48. The compound according to claim 38, wherein said spray drying is carried out using a nozzle atomizer.

49. The compound wherein in step b), said compound is being recovered in the form of substantially spherical beads.

50. A compound according to claim 38, wherein said phosphate is sodium, potassium, calcium, magnesium or a rare earth phosphate.

51. The compound according to claim 38, wherein said phosphate is a calcium phosphate, a monocalcium phosphate (MCP), a dicalcium phosphate (DCP) or a tricalcium phosphate (TCP).

52. The compound according to claim 51, wherein said calcium phosphate is a monocalcium phosphate (MCP) or a dicalcium phosphate (DCP).

53. The compound according to claim 38, having a phosphate content of at least 10% by weight.

54. The compound according to claim 53, having a phosphate content in the range 20% to 60% by weight.

55. The compound according to claim 38, having a tamped packing density (TPD) of more than 0.29.

56. The compound according to claim 38, having a DOP oil uptake of more than 170 ml/100 g.

57. The compound according to claim 38, having a DOP oil uptake higher than the one of a composition obtained by dry mixing said precipitated silica in the solid form and said phosphate in the solid form.

58. The compound according to claim 38, having a pore volume (V_{d1}) constituted by pores with a diameter of less than 1 μm, of at least 1.2 cm³/g.

59. The compound according to claim 38, having a BET specific surface of 60 m²/g to 250 m²/g.

60. The compound according to claim 38, having a Carr index of less than 0.1.

61. The compound according to claim 38, having:
a wear resistance R_{wr2} of at least 60, and/or a wear resistance R_{wr5} of at least 50%, and/or a wear resistance R_{wr10} of at least 15%.

62. The compound according to claim 38, being in the form of solid substantially spherical beads.

63. The compound according to claim 38, being in the form of substantially spherical non powdery beads.

64. The compound according to claim 38, being in the form of substantially spherical beads with a median diameter d₅₀ of at least 80 μm.

65. The compound according to claim 38, being a support further conditioned by a composition comprising at least one liquid absorbed on the support.

66. The compound according to claim 65, wherein said composition has a liquid content of at least 50% by weight.

67. The composition according to claim 65, wherein said liquid is a liquid additive, optionally a liquid animal foodstuff complement.

68. The composition according to claim 65, wherein said liquid is vitamin E, vitamin E acetate or choline hydrochloride.

69. A liquid animal foodstuff complement comprising the compound as defined in claim 65.

70. The liquid animal foodstuff complement according to claim 69, being simultaneously a nutritional additive for animals.

71. The liquid animal foodstuff complement according to claim 69, wherein said liquid is vitamin E, vitamin E acetate or choline hydrochloride.

72. An anticaking agent, comprising a compound as defined in claim 38, said compound optionally having been milled in advance.

73. A liquid atomization processing aid, a solid milling processing aid, a pelletizing or a tabletting aid, comprising a compound as defined in claim 38, said compound optionally having been milled in advance.
